(19) United States
(12) Patent Application Publication
(10) Pub. No.: US 2009/0204032 A1
(43) Pub. Date: Aug. 13, 2009

(54) LITHOTRIPSY APPARATUS

(76) Inventors: Norbert Herrmann, Ebnath (DE); Jochen Miguel Löseken, Bayreuth (DE)

Correspondence Address: BRINKS HOFER GILSON & LIONE, P.O. BOX 10395, CHICAGO, IL, 60610, US

(21) Appl. No.: 12/280,761

(22) PCT Filed: Feb. 1, 2007
(86) PCT No.: PCT/EP07/50961

§ 371 Date: Feb. 17, 2009

(30) Foreign Application Priority Data
Mar. 2, 2006 (DE) 10 2006 009 716.5

(51) Int. Cl. A61B 17/22; G01N 23/00; H05G 1/02

(52) U.S. Cl. 601/4; 378/1; 378/197

(57) ABSTRACT

A lithotripsy apparatus (2) comprises an X-ray system (3), with an isocentre (1) arranged in a mid-plane of the X-ray system (3), and a shockwave head (6) that can be oriented with respect to the isocentre (1) and is mounted on a support device (8). A particularly simple and stable mechanism for adjusting the shockwave head is obtained in which the support device (8) has a first adjustment unit (20) that can be moved linearly for horizontal adjustment of the shockwave head (6), a second adjustment unit (24) that can be moved linearly for adjusting the height of the shockwave head (6), and a rotary adjustment unit (22) for rotating the shockwave head (6).

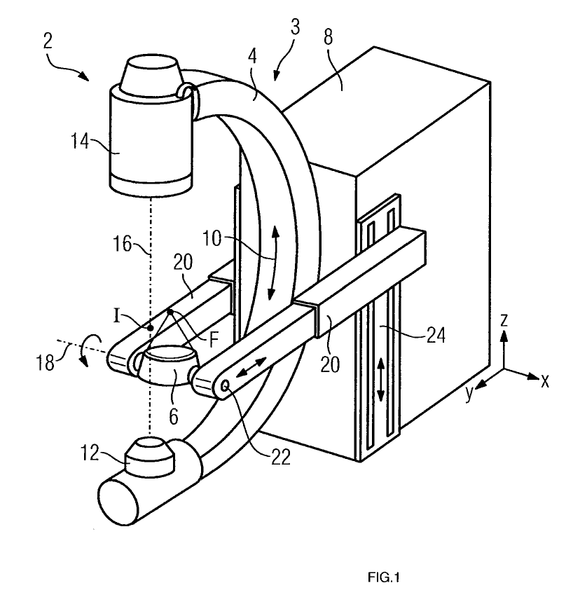

The invention relates to a lithotripsy apparatus comprising an X-ray system with an isocenter arranged in the mid-plane of the X-ray system and a shockwave head that can be oriented with respect to the isocenter and is mounted on a support device.

Lithotripsy is a medical procedure for extracorporeal fragmentation of calculi deposited in the kidneys or in the urinary tracts. A lithotripsy apparatus usually consists of an X-ray system for locating and visualizing the calculus, and a therapy system for breaking up the calculus. The X-ray usually includes an X-ray C-arm that can be moved orbitally around an isocenter. An X-ray source and an image amplifier are arranged at the ends of the arms of the X-ray C-arm. When in use, the C-arm partially encompasses a patient table, so that the calculus lies in the isocenter. The therapy system includes a shockwave head by means of which shockwaves (sound pressure waves) are generated. In order to largely avoid damage to the tissue surrounding the calculus, it is necessary to position the focal point of the shockwave head in the calculus or in the isocenter of the X-ray C-arm.

A lithotripsy apparatus in which the shockwave head is supported by an articulated arm is known from DE 103 42 016 A1. The articulated arm has three axes of rotation or three pivot joints parallel to one another, by which the shockwave head is positioned.

The object underlying the invention is to specify a lithotripsy apparatus that is characterized by a rigid form of construction and a simple adjusting mechanism.

The object is achieved according to the invention by a lithotripsy apparatus comprising an X-ray system with an isocenter arranged in a mid-plane of the X-ray system, and a shockwave head that can be oriented with respect to the isocenter and is mounted on a support device, it being possible for the support device to have a first linearly-movable adjustment unit for horizontal adjustment of the shockwave head, a second linearly-movable adjustment unit for adjusting the height of the shockwave head, and a rotary adjustment unit for a rotation of the shockwave head.

The invention allows considerable simplification of the mechanical support and traversing system of the shockwave head in which the positioning of the shockwave head is now effected via two translational movements and one rotational movement. This facilitates a particularly simple and rugged construction of the linearly-movable adjustment units, which results in a high degree of stiffness of the support device and therefore of the entire lithotripsy apparatus. In addition, because of its ability to rotate by up to 360°, the shockwave head can be maneuvered in numerous operating positions, thus ensuring maximum treatment accuracy.

Viewed in a Cartesian coordinate system, the height adjustment of the shockwave head is effected along a z-axis and the horizontal adjustment along a y-axis. The rotational movability of the shockwave head occurs about a rotational axis which runs parallel to the x-axis of the coordinate system. The X-ray system is supported so that it makes no movement along an x-axis. A horizontal adjustment of the shockwave head in the x-direction is therefore unnecessary. In this case the mid-plane is defined by the z-y plane in which the isocenter lies.

According to a preferred embodiment, the linearly-movable adjustment units are designed in such a way that the movement for the height adjustment is decoupled from the movement for the horizontal adjustment of the shockwave head. The advantage of this embodiment is that the traversing of the shockwave head is effected via axes which are mechanically independent of each other, so that the forces due to the weight of the shockwave head, which affect the first linear adjustment unit, are transmitted over a very short path in the support device. Moreover, it is advantageous that the movements that are decoupled from each other enable particularly simple control of the adjustment units.

According to a further preferred embodiment, the linearly-movable adjustment units are perpendicular to each other. From a constructional point of view this embodiment is particularly simple to realize. Furthermore, this therefore ensures that during the adjustment of one of the adjustment units, traversing of the shockwave head takes place exclusively in the horizontal or the vertical direction. This makes it particularly easy to control the adjustment units.

The X-ray system is preferably supported on the support device. This facilitates a particularly compact form of construction of the lithotripsy apparatus. In this case the positions of the X-ray system are mechanically determined relative to the shockwave head, so that no extensive calculations or locating processes are required in order to determine and vary the position of the focal point of the shockwave head in relation to the isocenter.

In order to achieve particularly accurate focusing of the shockwaves in the isocenter, the X-ray system is supported on the support device in such a way that the focal point of the shockwave head lies in the mid-plane. Therefore in no way will a displacement of the isocenter within the mid-plane impair the focusing accuracy of the shockwave head. In particular, this enables an orbital movability of the X-ray system to be achieved, which facilitates convenient detection and monitoring of the calculus from different directions. The isocenter remains in the mid-plane in every position along the orbital traverse path, as does the focal point of the shockwave head and the shock waves can be precisely oriented with respect to the isocenter.

A particularly high stability is achieved for the first linearly-movable adjustment unit in that this is preferably constructed from two adjustment arms between which the shockwave head is supported in a rotatable manner. Due to the two adjustment arms, a mirror-symmetrical embodiment of the support of the shockwave head is achieved, which results in very high stability. In particular, this prevents deformation of the adjustment unit or even lateral tilting of the apparatus due to asymmetrical loading. The shockwave head is maintained in a particularly stable position and the force with which it acts on the adjustment arms is transferred on both sides along the shortest path to the support device.

Usefully, the adjustment arms are of a telescopic design. The telescopic in and out movement of the adjustment arms offers a space-saving solution.

Further conventional and proven structural components are used in order to ensure simple movability of the second linearly-movable adjustment unit. Advantageously, the second linearly-movable adjustment unit includes two guides which are arranged separately from each other on the support device. The guides, which are in particular linear guides, ensure movement of the shockwave head only in the vertical direction and consequently the height adjustment is decoupled from the horizontal movement of the shockwave head. The guide/drive system is embodied, in particular, as a type of vertically oriented recirculating ball screw which is characterized by low friction and wear.

In order to ensure a particularly simple coupling between the first and the second adjustment unit, each of the adjustment arms is usefully retained in one of the guides. In addition, each of the adjustment arms is supported in particular in a plurality of points or linearly in the guides, so that a particularly rigid construction exists in which the weight of the shockwave head acting at the other end of the adjustment arm causes negligibly little bending of the adjustment arm.

### BRIEF DESCRIPTION OF THE DRAWING

A variant of the lithotripsy apparatus is explained in further detail with the aid of a drawing. Here the single FIGURE shows a perspective representation of a lithotripsy apparatus with one rotary and two linearly-movable adjustment units.

### DETAILED DESCRIPTION

A lithotripsy apparatus 2 for extracorporeal fragmentation of a calculus, for example a kidney stone, is illustrated in the FIGURE. The lithotripsy apparatus 2 includes an X-ray system 3 with an X-ray C-arm, 4, a shockwave head 6 and a support device 8. Both the C-arm 4 and the shockwave head 6 are supported on the support device 8. The C-arm 4 is orbitally movable and its traversing direction is indicated by the arrow 10. An X-ray source 12 and an imaging unit 14, which are likewise part of the X-ray system 3, are arranged at the ends of the limbs of the X-ray C-arm 4. In this exemplary embodiment the imaging unit 14 includes an image amplifier and other components for detecting an X-ray beam penetrating the calculus and an image of the calculus. A beam axis 16 which contains the isocenter I of the X-ray C-arm 4, passes through the X-ray source 12 and the imaging unit 14. During the operation of the lithotripsy apparatus 2, the C-arm 4 encompasses a patient table—not shown here—on which the patient is supported. In the course of this the calculus to be fragmented is precisely positioned in the isocenter I.

The y-z plane in which the beam axis 16 lies is the mid-plane which contains the isocenter I. The shockwave head 6 is attached to the support device 8 in such a way that its focal point F is likewise always located in the mid-plane. Consequently, during therapy the focal point F is positioned at the isocenter I merely via rotation and traversing of the shockwave head 6 in the mid-plane.

The fragmentation of the calculus is achieved by means of focused sound waves/shockwaves that are generated by the shockwave head 6. The generated shock waves are focused at a focal point F which during the treatment coincides with the isocenter I, in which the calculus also lies, in order to avoid damage to the tissue outside the calculus.

In order to bring the focal point F into the correct position, a translational movement of the shockwave head 6 along the z and y axes, and a rotation about an axis of rotation 18 parallel to the x-axis of a Cartesian coordinate system is facilitated.

The horizontal adjustment of the shockwave head 6 is effected via a linearly-movable adjustment unit 20, which in this exemplary embodiment is designed in the form of two adjustment arms. The adjustment arms 20 are supported on the support device 8 and can be moved in and out telescopically via a drive system. The forces acting on the adjustment unit 20 are thus symmetrically distributed, so that the loading of each adjustment arm 20 is low. Moreover, these forces are transferred to the support device 8 over a particularly short linear path.

The shockwave head 6 is retained in a rotatable manner between the two adjustment arms via a rotary adjustment unit 22, which in this embodiment consists of two pivot bearings 22 and a shaft, not shown. In this case the shockwave head 6 can rotate by up to 360° about an axis of rotation 18 passing through the pivot bearings. The rotational bearing arrangement of the shockwave head 6 is provided in particular by means of a gear, for example a planetary gear or a harmonic drive gear. In order to lock the shockwave head 6 in the desired position a locking element, for example a brake shoe, is provided—not shown in this exemplary embodiment—which is pressed against the shaft to hold the shockwave head 6. The electrical energy required to generate the shock wave is conducted into the rotary shockwave head 6 in the region of the pivot bearing 22, for example via a slip ring or a liquid metal connection.

At its other end, each adjustment arm 20 is attached at two points to the support device 8 via a guide/drive system with linear guides 24. The guides 24 are arranged vertically to the adjustment arms 20 and enable the height of the adjustment arms 20 or of the shockwave head 6 to be adjusted. The guides 24 on each side of the support device 8 are, in particular, elements of a motor-driven recirculating ball screw—not shown here—which essentially has a vertically oriented thread and a screw nut. The adjustment arms 20 are attached to the screw nut and simultaneously supported via the guides 24. The rotation of the thread is converted into a translational movement of the nut, thereby positioning the adjustment arms 20 in the vertical direction.

Due to the type of support of the adjustment arms 20, the height adjustment of the shockwave head 6 is decoupled from its horizontal adjustment, that is to say the vertical traversing of the shockwave head 6 occurs independently of the outwards or inwards movement of the adjustment arms 20. The rotation of the shockwave head 6 is likewise effected independently of the translational movements of the adjustment arms 20. The shockwave head 6 is thus traversed via mechanically independent axes, which considerably simplifies the control of the movement sequence.

1. A lithotripsy apparatus comprising:
an X-ray system with an isocenter arranged in a mid-plane of the X-ray system; and
a shockwave head that can be oriented with respect to the isocenter and is mounted on a support device,
wherein the support device includes a first linearly-movable adjustment unit for horizontal adjustment of the shockwave head, a second linearly-movable adjustment unit for adjusting the height of the shockwave head, and a rotary adjustment unit for rotating the shockwave head.

2. The lithotripsy apparatus as claimed in claim 1, wherein the linearly-movable adjustment units are designed such that the movement for the height adjustment is decoupled from the movement for the horizontal adjustment of the shockwave head.

3. The lithotripsy apparatus as claimed in claim 1, wherein the linearly-movable adjustment units are perpendicular to each other.

4. The lithotripsy apparatus as claimed in claim 1, wherein the X-ray system is supported on the support device.

5. The lithotripsy apparatus as claimed in claim 4, wherein the X-ray system is supported on the support device in such a way that a focal point of the shockwave head lies in the mid-plane.

6. The lithotripsy apparatus as claimed in claim 1, wherein the first linearly-movable adjustment unit includes first and second adjustment arms between which the shockwave head is supported in a rotatable manner.

7. The lithotripsy apparatus as claimed in claim 6, wherein the first and second adjustment arms are telescopic.

8. The lithotripsy apparatus as claimed in claim 6, the second linearly-movable adjustment unit includes first and second guides, which are arranged separately from each other on the support device.

9. The lithotripsy apparatus as claimed in claim 8, wherein the first adjustment arms is retained in the first guide and the second adjustment arm is retained in the second guide.

10. The lithotripsy apparatus as claimed in claim 2, wherein the linearly-movable adjustment units are perpendicular to each other.

11. The lithotripsy apparatus as claimed in claim 10, wherein the X-ray system is supported on the support device.

12. The lithotripsy apparatus as claimed in claim 11, wherein the X-ray system is supported on the support device in such a way that a focal point of the shockwave head lies in a mid-plane.
